(19) United States
(12) Patent Application Publication
(10) Pub. No.: US 2007/0141094 A1
(43) Pub. Date: Jun. 21, 2007

(54) Composite of foamed substance and soap substance

(76) Inventor: Jone Chang, Taipei City (TW)

Correspondence Address: BIRCH STEWART KOLASCH & BIRCH, PO BOX 747, FALLS CHURCH, VA, 22040-0747, US

(21) Appl. No.: 11/312,449

(22) Filed: Dec. 21, 2005

(51) Int. Cl. A61K 8/02

(52) U.S. Cl. 424/401

(57) ABSTRACT

A composite of foamed substance and soap substance includes a foamed substance formed according to a selected shape and encased by a soap substance. When the soap substance is almost totally consumed, it can still attach to the foamed substance so that it can be fully used. After the soap substance is exhausted, the foamed substance can still be used continuously. The foamed substance has a fiber structure which can be rubbed to easily generate a greater amount of fine foams to save consumption of the soap substance and achieve a cleaning effect. The foamed substance is formed by interweaving of foamed substance fibers which have a surface covered by a resin layer. The resin layer is elastic and can enhance the extensibility of the foamed substance, and can also be permeated with a water-base pigment to enhance the aesthetic and visual appealing of the entire product.

### FIELD OF THE INVENTION

The present invention relates to a composite of foamed substance and soap substance and particularly to a composite that has a foamed substance encased in a soap substance.

### BACKGROUND OF THE INVENTION

Soaps are generally used by people to clear dirt from body or articles. The conventional soaps mostly are made from the same material and duffer mainly in the shape after solidifying. While the soap can generate a desirable amount of foams at the initial stage, after being used for a period of time and the size becomes smaller, it is difficult to be grasped and the foam generating property deteriorates.

The soap may also be coupled with various materials to become new composites and versatile products to make using more convenient. For instance, U.S. Pat. No. 4,062,792 entitled “Soap cake construction and manufacture” has a support plate embedded in the soap to facilitate user holding when in use. The soap also may be encased by an elastic cover to facilitate foam generation.

U.S. Pat. No. 4,335,007 entitled “Soap bar” has a base made of water absorbing plastics or insoluble material to facilitate water dispelling. However, as the soap is soluble, after used for a period of time, the soap and the base are separated, and the original function is lost.

U.S. Pat. No. 5,869,437 entitled “Transparent soap with dissolvable logo” contains a decomposable polymer material printed with trademarks, patterns or information. The polymer material is embedded in the transparent soap to achieve commercial effect.

U.S. Pat. No. 6,171,007 entitled “Washing cake of soap and its fabrication method” has a net and liquid soap material formed in a mold. After the liquid soap material is solidified, the net is embedded in the soap. When the soap is in contact with water and consumes gradually, the elastic net is exposed outside the soap surface and can generate foams during scrubbing. As the elastic net is rough and has relative large holes, scrubbing user's skin to produce foams often generates a uncomfortable feeling and the foams cannot be produced at a greater amount rapidly.

### SUMMARY OF THE INVENTION

The primary object of the present invention is to solve the aforesaid disadvantages. The present invention provides a composite that has foamed substance encased in soap substance. It includes a foamed substance made according to a preset form and a soap substance to encase the foamed substance. As the foamed substance is encased in the soap substance, separation of the soap substance and the foamed substance can be prevented.

Furthermore, as the foamed substance is embedded in a selected location of the soap substance, it can compensate foam degeneration problem of after the soap substance is used for a period of time. Hence even if the soap substance is almost consumed completely, it can still attach to the foamed substance until it is fully used. After the soap substance is fully consumed, the foamed substance still can be used continuously. The foamed substance is formed by interweaving of fibers of the foamed substance. Such a fiber structure can produce a greater amount of fine foams when rubbed. Thus it can save the consumption of the soap substance and generate cleaning effect by scrubbing lightly. Moreover, the fiber structure of the foamed substance forms a mesh structure, and the surface of the foamed fiber structure is covered by a resin layer. The resin layer is elastic and can enhance the extensibility of the foamed substance. Therefore wearing of the foamed substance caused by rubbing and scrubbing can be reduced. In addition, the resin layer can be permeated with a water-based pigment to enhance the aesthetic and visual appealing of the entire product. With the protection of the resin layer, the color of the pigment does not fade.

In another aspect, the soap substance and the foamed substance can be threaded by a rope to facilitate user grasping and holding when in use.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a perspective view of an embodiment of the present invention.

FIG. 2 is a sectional view of an embodiment of the present invention.

FIG. 3 is a schematic view of an embodiment of the present invention.

FIG. 4 is a schematic view of another embodiment of the present invention.

### DETAILED DESCRIPTION OF THE PREFERRED EMBODIMENTS

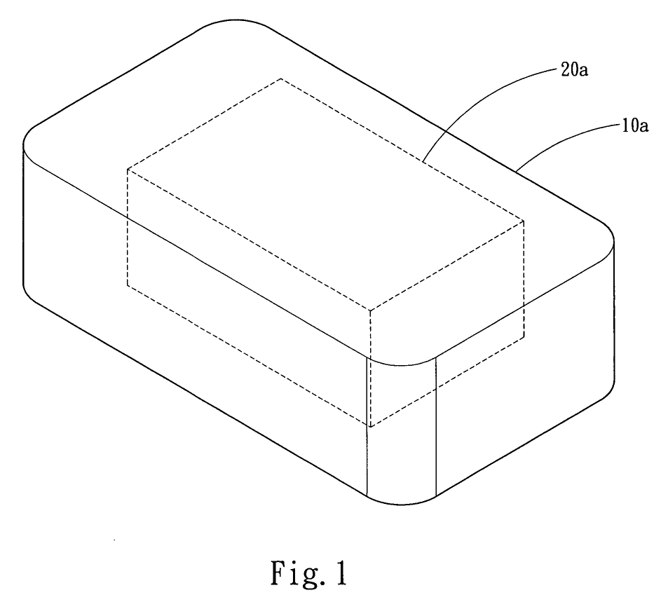

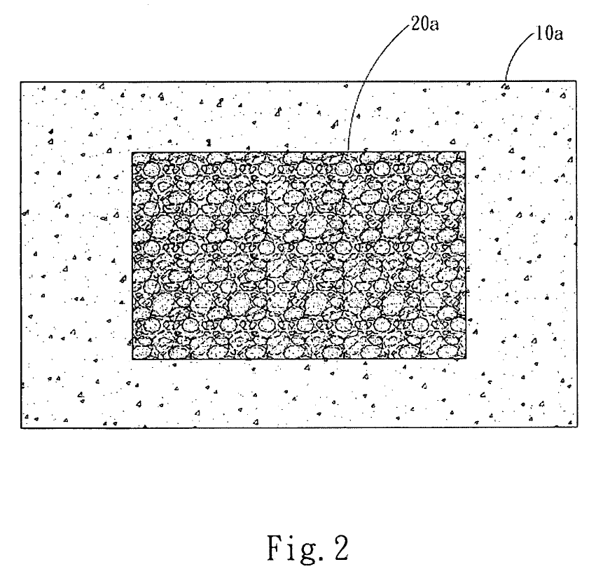

Please refer to FIGS. 1 and 2 for an embodiment of the invention. The composite of foamed substance and soap substance of the invention includes a foamed substance 20a and a soap substance 10a. First, the soap substance 10a is melted; next, the foamed substance 20a is formed in a selected shape and placed in the melting soap substance 10a; then the soap substance 10a is solidified to encase the foamed substance 20a.

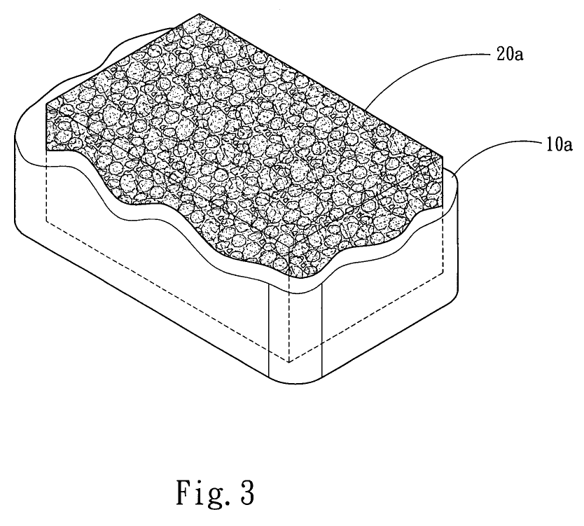

Referring to FIG. 3, the foamed substance 20a is encased in the soap substance 10a. Hence the problem of separation of the soap substance 10a and the foamed substance 20a can be prevented. Moreover, as the foamed substance has crevices, the molten soap substance 10a can seep in the crevices of the foamed substance 20a. When the soap substance 10a is almost totally consumed, it can still attach to the foamed substance 20a, hence can be fully used. When the soap substance 10a is exhausted, the foamed substance 20a can still be used continuously. On the other hand, when users scrub the body or other objects with the invention, the fiber structure of the foamed substance 20a can be rubbed to generate a greater amount of fine foams. Thus consumption of the soap substance 10a can be reduced and cleaning effect can still be maintained. In addition, the foamed substance 20a is constructed by interweaving of foamed substance fibers, and the surface of the foamed substance fibers is covered by a resin layer (not shown in the drawings) which is elastic. Hence the extensibility of the foamed substance can be enhanced. Thereby when the foamed substance 20a is used for scrubbing and stretched, the possibility of its being damaged also decreases. Thus breaking of the foamed substance 20a to small pieces during rubbing and scrubbing can be prevented. In addition, the resin layer can provide an improved friction interface for the composite to make scrubbing on the skin smoother. The resin layer can also be permeated with a water-based pigment of different colors. The water-based pigment is contained in the resin layer, thus the color do not fade and spotty coloring can be avoided. This helps to enhance the aesthetic and visual appealing of the entire product.

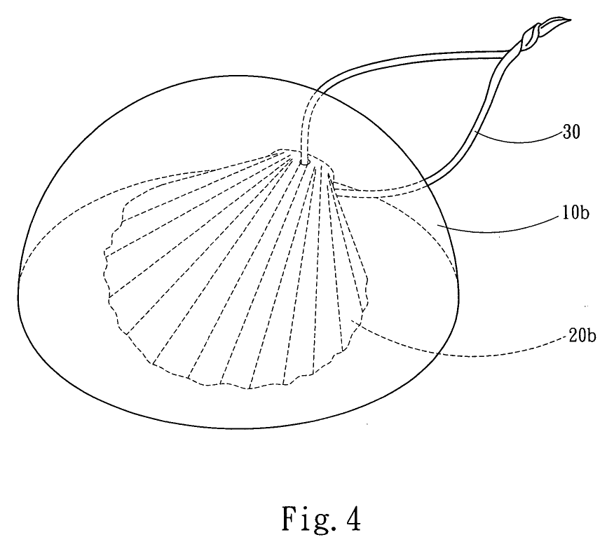

Refer to FIG. 4 for another embodiment of the invention. The molten soap substance 10b can be poured into different molds to form varying shapes, and the foamed substance 20b can also be formed in various shapes and embedded in the soap substance 10b to produce the composite with versatile designs. In addition, the soap substance 10b and the foamed substance 20b may be threaded by a rope 30 to facilitate user grasping and holding when in use.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A composite of foamed substance and soap substance, comprising:
a foamed substance produced according to a selected shape; and
a soap substance to encase the foamed substance.

2. The composite of foamed substance and soap substance of claim 1, wherein the foamed substance is constructed by interweaving foamed substance fibers which form a mesh structure and have a surface covered by a resin layer which is elastic and colored.

3. The composite of foamed substance and soap substance of claim 2, wherein the resin layer is permeated with a water-based pigment.

4. The composite of foamed substance and soap substance of claim 1, wherein the soap substance and the foamed substance are threaded by a rope.
